(19) United States
(12) Patent Application Publication
(10) Pub. No.: US 2005/0251196 A1
(43) Pub. Date: Nov. 10, 2005

(54) Surgical tool for use in expanding a tubular structure

(75) Inventor: Chi Yin Wong, Canton, MA (US)

Correspondence Address: TAROLLI, SUNDHEIM, COVELL & TUMMINO L.L.P., SUITE 1111, 526 SUPERIOR AVENUE, CLEVELAND, OH, 44114-1400, US

(73) Assignee: Endius Incorporated

(21) Appl. No.: 10/839,830

(22) Filed: May 6, 2004

(51) Int. Cl. A61M 29/00

(52) U.S. Cl. 606/198

(57) ABSTRACT

A surgical tool for use in expanding a tubular structure includes a housing. First and second legs movable relative to the housing have first and second ends engageable with an inner surface of the tubular structure. A handle movable relative to the housing moves the first and second ends away from each other to apply a radially outwardly directed force to the inner surface of the tubular structure and cause expansion of the tubular structure. A stop member is connected with the housing. The handle is engageable with the stop member to limit movement of the first and second ends away from each other. The stop member has a first position relative to the housing to limit movement of the first and second ends away from each other a first predetermined distance. The stop member has a second position relative to the housing to limit movement of the first and second ends away from each other a second predetermined distance smaller than the first predetermined distance.

### FIELD OF THE INVENTION

The present invention relates to a surgical tool for use in expanding a tubular structure or cannula for receiving surgical instruments to perform a surgical procedure on a body.

### BACKGROUND OF THE INVENTION

Minimally invasive surgical techniques allow a surgical procedure to be performed on a patient's body through a relatively small incision in the body and with a limited amount of body tissue disruption. Minimally invasive surgery typically utilizes a tubular structure known as a cannula or retractor which is inserted into a small incision in the body. The cannula or retractor retracts tissue and holds the incision open to serve as a conduit extending between the exterior of the body and the local area inside the body where the surgery is to be performed.

### SUMMARY OF THE INVENTION

The present invention is a surgical tool for use in expanding a tubular structure. The tubular structure has an inner surface defining a passage through the tubular structure for receiving surgical instruments. The surgical tool includes a housing. A first leg movable relative to the housing has a first end engageable with the inner surface of the tubular structure. A second leg movable relative to the housing has a second end engageable with the inner surface of the tubular structure. At least one handle movable relative to the housing moves the first and second ends away from each other to apply a radially outwardly directed force to the inner surface of the tubular structure and cause expansion of the tubular structure to increase a cross-sectional area of the passage along a portion of the passage. A stop member is connected with the housing. The handle is engageable with the stop member to limit movement of the first and second ends away from each other. The stop member has a first position relative to the housing to limit movement of the first and second ends away from each other a first predetermined distance. The stop member has a second position relative to said housing to limit movement of the first and second ends away from each other a second predetermined distance smaller than the first predetermined distance.

### BRIEF DESCRIPTION OF THE DRAWINGS

The foregoing and other features of the present invention will become apparent to one skilled in the art to which the present invention relates upon consideration of the following description of the invention with reference to the accompanying drawings, wherein:

FIG. 1 is a perspective view of a surgical tool constructed in accordance with the present invention;

FIG. 2 is an exploded perspective view of the surgical tool of FIG. 1;

FIG. 3 is an enlarged exploded view of a portion of the surgical tool of FIG. 1;

FIG. 4 is a cross-sectional view of the surgical tool of FIG. 1;

FIG. 5 is an enlarged cross-sectional view of a portion of the surgical tool of FIG. 1; and

FIG. 6 is an exploded view of a depth limiter of the surgical tool of FIG. 1;

FIG. 7 is an exploded perspective view of a tubular structure or cannula that is expandable by the surgical tool, the cannula being shown in an expanded condition; and

FIG. 8 is a perspective view of the cannula of FIG. 7 with parts removed for clarity, the cannula being shown in a contracted condition.

### DESCRIPTION OF THE INVENTION

The present invention is directed to a surgical tool for use in expanding a tubular structure or cannula for performing a surgical procedure on the body of a patient. The surgical tool may be used to expand a variety of tubular structures or cannulae.

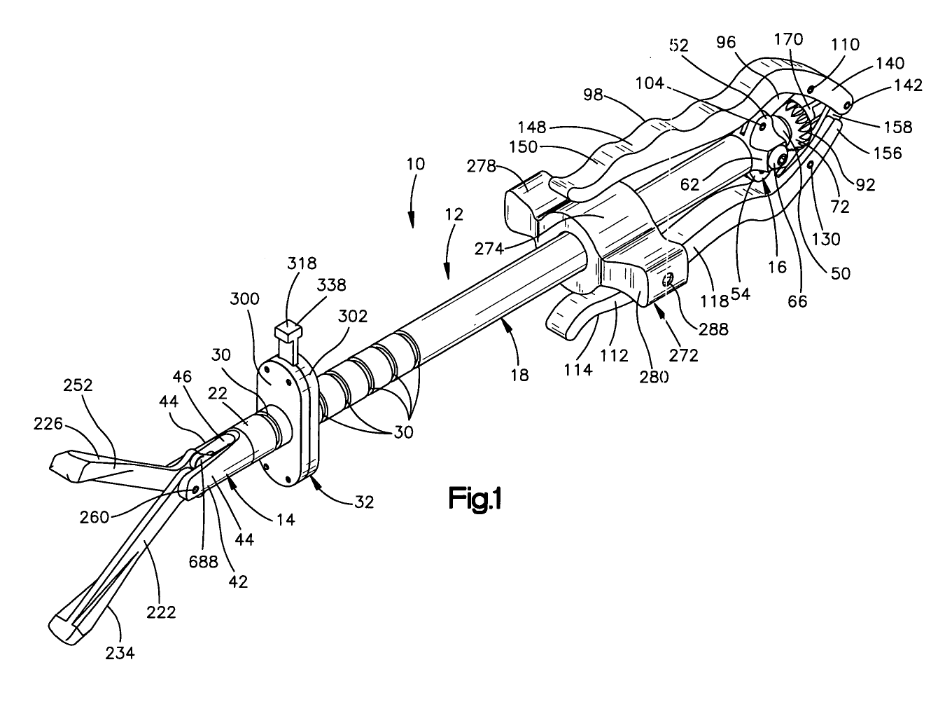

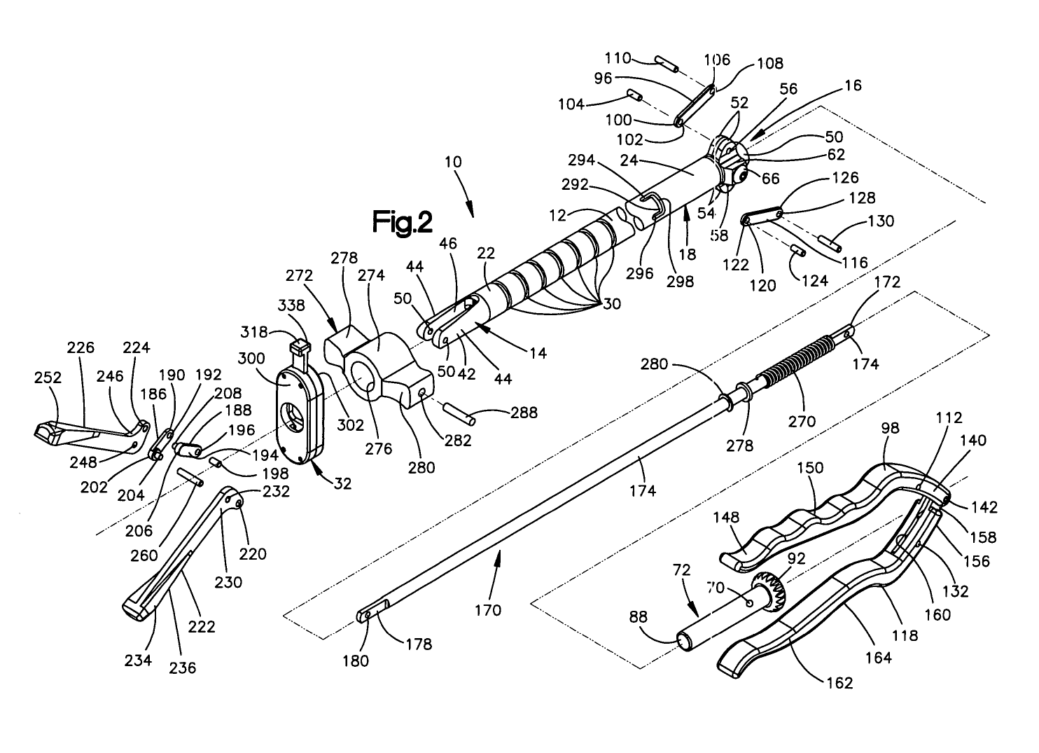

A surgical tool 10 constructed according to the present invention is illustrated in FIGS. 1-6. The surgical tool 10 (FIGS. 1-2) includes a tubular housing 12. The housing 12 has a tubular first or distal member 14 and a tubular second or proximal member 16. A tubular intermediate member 18 interconnects the distal and proximal members 14 and 16. The distal and proximal members 14 and 16 are welded to the intermediate member 18. It is contemplated that the proximal and distal members 14 and 16 may be connected to the intermediate member 18 in any suitable manner.

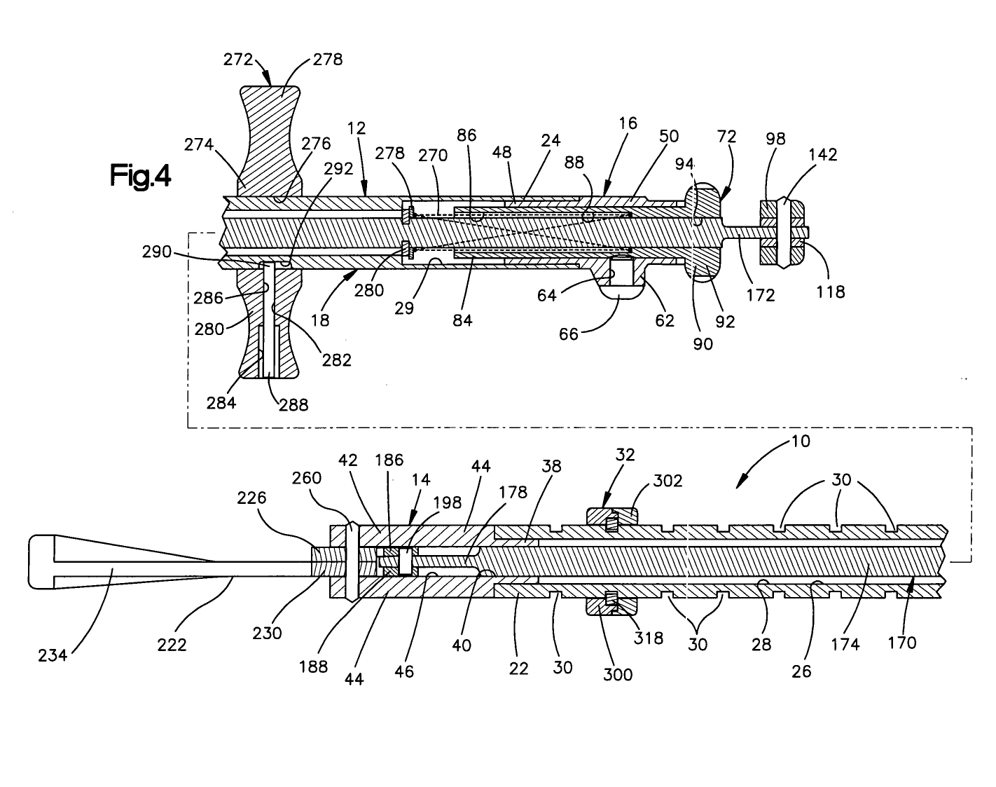

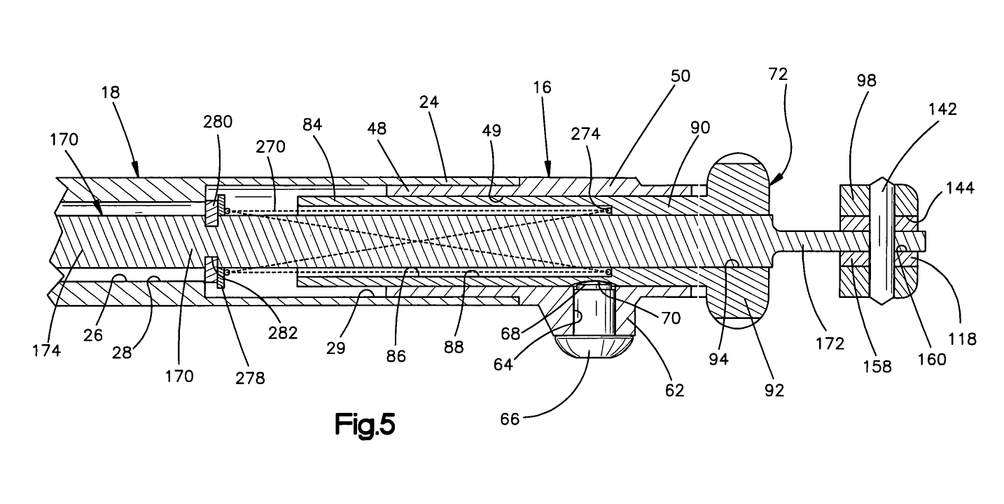

The intermediate member 18 (FIG. 2) includes a first or distal end portion 22 connected to the distal member 14. The intermediate member 18 has a second or proximal end portion 24 connected to the proximal member 16. The intermediate portion 18 (FIGS. 4-5) is tubular and defines a passage 26 extending through the intermediate member. The passage 26 has a first portion 28 with a first diameter and a second portion 29 in the second end portion 24 with a second diameter larger than the first diameter.

The intermediate member 18 includes a plurality of annular grooves 30 (FIGS. 1-2) in the outer surface that define a plurality of positions for connecting a depth limiter 32 to the intermediate member 18. Although the intermediate member 18 is shown with seven annular grooves 30, it is contemplated that the intermediate member may have any number of annular grooves. The depth limiter 32 engages a proximal end of the tubular structure to limit the depth that the surgical tool 10 extends into the tubular structure. Accordingly, the depth limiter 32 is placed in a desired position on the intermediate member 18 depending on the length of the tubular structure.

The distal member 14 (FIG. 4) includes a first end portion 38 that extends into the passage 26 in the intermediate member 18 to connect the distal member with the intermediate member. The first end portion 38 of the distal member 14 is tubular and has an outer diameter substantially equal to the diameter of the first portion 28 of the passage 26. The first end portion 38 defines a passage 40 which is a continuation of the passage 26.

The distal member 14 (FIGS. 1-2) includes a second end portion 42 having an outer diameter equal to the outer diameter of the intermediate member 18. The second end portion 42 includes a pair of axially extending projections 44. The projections 44 extend generally parallel to each other and define a channel 46 between them. The channel 46 is a continuation of the passage 40 in the first end portion 38. Each of the projections 44 (FIG. 2) has a through-hole 50 that intersects the channel 46.

The proximal member 16 (FIG. 5) includes a first end portion 48 that extends into the second portion 29 of the passage 26 in the second end portion 24 of the intermediate member 18. The first end portion 48 is tubular and has an outer diameter substantially equal to the diameter of the second portion 29 of the passage 26. The first end portion 48 defines a passage 49 which is a continuation of the passage 26.

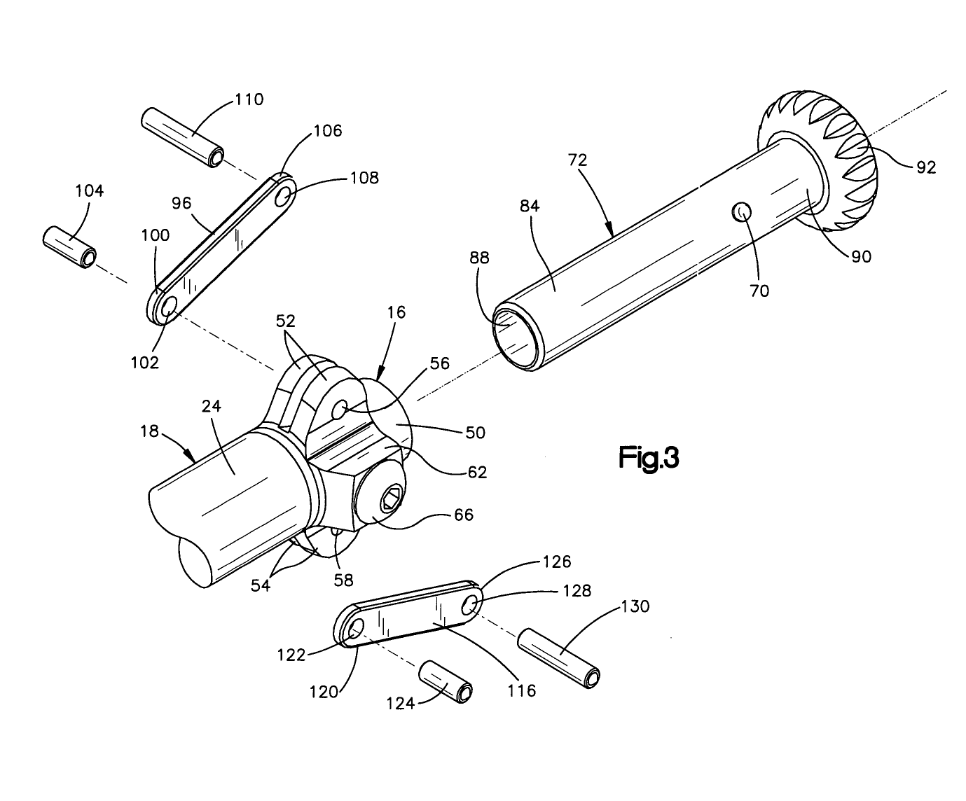

The proximal member 16 (FIGS. 1-3) includes a second end portion 50 that extends from the first end portion 48. The second end portion 50 includes a pair of radially extending flanges 52. A pair of radially extending flanges 54 extend from the second end portion 50 opposite the flanges 52. Each of the flanges 52 has a through-hole 56, one of which is shown in FIG. 3. Each of the flanges 54 has a through-hole 58, one of which is shown in FIG. 3.

A radially extending projection 62 extends generally perpendicular to the flanges 52 and 54. The radially extending projection 62 (FIG. 5) has a radially extending threaded opening 64. The opening 64 intersects the passage 49 in the proximal member 16.

A fastener or screw 66 threadably engages the opening 64. The screw 66 has an end portion 68 that extends into a circular recess 70 in a sleeve 72. The screw 66 connects the sleeve 72 to the housing 12 and prevents movement between the sleeve and the housing it is contemplated that the sleeve 72 may be connected to the housing 12 in any suitable manner.

A first tubular end portion 84 (FIG. 5) of the sleeve 72 extends into the passage 49 of proximal member 16. The end portion 84 has an outer diameter that is smaller than the diameter of the passage 49 in the proximal member 16. The end portion 84 defines a larger diameter portion 86 of a passage 88 through the sleeve 72. A second end portion 90 (FIGS. 3-5) of the sleeve 72 includes a radially extending portion 92. The second end portion 90 (FIG. 5) defines a smaller diameter portion 94 of the passage 88.

A first link 96 (FIGS. 2-3) is pivotally connected to the flanges 52 on the proximal member 16 and to a first handle 98. The link 96 has a first end 100 with a through-hole 102. The first end 100 of the link 96 extends between the flanges 52. A pivot pin 104 extends through the through-hole 102 and the through-holes 56 in the flanges 52 to pivotally connect the link 96 to the flanges 52. A second end 106 of the link 96 has a through-hole 108. A pivot pin 110 extends through through-holes 112 in the handle 98 and the through-hole 108 to pivotally connect the link 96 to the handle 98.

A second link 116 is pivotally connected to the flanges 54 on the proximal member 16 and to a second handle 118. The link 116 has a first end 120 with a through-hole 122. The first end 120 of the link 116 extends between the flanges 54. A pivot pin 124 extends through the through-holes 58 in the flanges 54 and the through-hole 122 to pivotally has a through-hole 128. A pivot pin 130 extends through through-holes 132 in the handle 118 and the through-hole 128 to pivotally connect the link 116 to the handle 118.

The first handle 98 (FIGS. 1-2) has a first end portion 140 pivotally connected to the second handle 118 by a pivot pin 142. The first end portion 140 of the handle 98 has a channel 144 (FIG. 5) into which the second handle 118 extends. The pivot pin 142 extends through through-holes in the first end portion 140 that intersect the channel 144 and a through-hole in the second handle 118 to pivotally connect the first and second handles 98 and 118. The second end portion 106 (FIG. 2) of the link 96 is also received in the channel 144. The through-holes 112 in the handle 98 intersect the channel 144 and receive the pivot pin 110 to pivotally connect the link 96 to the handle 98.

The handle 98 (FIGS. 1-2) includes a second end portion 148 extending axially from the first end portion 140. The second end portion 148 extends in a distal direction from the first end portion 140. The second end portion 148 of the handle 98 has gripping features located on an upper surface 150.

The second handle 118 (FIG. 2) has a first end portion 156 pivotally connected to the first end portion 140 of the first handle 98. The first end portion 156 has a portion 158 with a width less than the width of the channel 144 that extends into the channel 144 in the first handle 98. The pivot pin 142 extends through through-holes in the first handle 98 and a through-hole in the portion 158 of the second handle 118 to pivotally connect the handles to each other.

The first end portion 156 (FIG. 2) of the second handle 118 also includes a channel 160. The second end portion 126 of the link 116 extends into the channel 160. The through-holes 132 in the second handle 118 intersect the channel 160 and receive the pivot pin 130 to pivotally connect the link 116 to the second handle 118.

The second handle 118 (FIGS. 1-2) includes a second end portion 162 extending axially from the first end portion 156. The second end portion 162 extends in a distal direction from the first end portion 156. The second end portion 162 of the handle 118 has gripping features located on a lower surface 164.

The first end portions 140 and 156 (FIGS. 2 and 5) of the handles 98 and 118 are also pivotally connected to an actuator 170 by the pivot pin 142. The actuator 170 has a first flattened end portion 172 with a through-hole 174. The first end portion 172 of the actuator 170 extends into the channel 160 in the handle 118. The pivot pin 142 extends through the through-hole 174 in the actuator 170 and the through-holes in the handles 98 and 118 to pivotally connect the actuator to the handles.

The actuator 170 (FIGS. 4-5) extends axially through the passage 88 in the sleeve 72, the proximal member 16, and the passage 26 in the intermediate member 18 into the channel 46 in the distal member 14. The actuator 170 has a cylindrical central portion 174 extending between the first flattened end portion 172 and a second flattened end portion 178 that extends into the channel 46. The central portion 174 has a diameter smaller than the smaller diameter portion 94 of the passage 88 in the sleeve 72 and smaller than the diameter of the passage 40 in the distal member 14 to permit axial movement of the actuator 170.

The second end portion 178 (FIG. 2) of the actuator 170 has a through-hole 180 for pivotally connecting the second end portion 178 with linking members 186 and 188. The second end portion 178 extends between the linking members 186 and 188. The first linking member 186 has a first end portion 190 with a through-hole 192. The second linking member 188 has a first end portion 194 with a through-hole 196. A pivot pin 198 extends through the through-holes 192 and 196 in the linking members 186 and 188 and through the through hole 180 in the actuator 170 to pivotally connect the linking members to the actuator.

The linking member 186 has a second end portion 202 with a cylindrical portion 204 extending toward the linking member 188. The second linking member 188 has a second end portion 206 with a cylindrical portion 208 extending toward the first linking member 186. The cylindrical portion 204 on the first linking member 186 extends into a through-hole 220 in a first leg or jaw 222 to pivotally connect the leg to the first linking member 186. The cylindrical portion 208 on the second linking member 188 extends into a through-hole 224 in a second leg or jaw 226 to pivotally connect the second leg 226 to the linking member 188.

The jaw 222 has a first end 230 through which the through-hole 220 extends. A second through-hole 232 extends through the first end 230 of the jaw 222. A second end 234 of the leg or jaw 222 extends from the first end 230. The second end 234 has a radial width greater than the width of the first end 230.

The second leg or jaw 226 has a first end 246 through which the through-hole 224 extends. The first end 246 also includes a through-hole 248. A second end 252 of the leg 226 extends from the first end 246. The second end 252 has a radial width which is greater than the radial width of the first end 246.

The first ends 230 and 246 of the legs or jaws 222 and 226 extend into the channel 46 between the projections 44. A pivot pin 260 extends through the through-holes 248 and 232 in the first and second legs 222 and 226. The pivot pin 260 also extends into the through-holes 50 in the distal member 14 to pivotally connect the legs 222 and 226 to each other and the distal member 14. Accordingly, axial movement of the actuator 170 relative to the housing 12 causes pivotal movement of the legs 222 and 226 relative to the distal member 14.

The actuator 170 (FIGS. 2 and 4-5) extends through a spring 270 located in the portion 86 of the passage 88 in the sleeve 72. The spring 270 extends from a shoulder 274 on the sleeve 172 to a washer 278 on the actuator 170. A C-shaped snap ring 280 extends into an annular groove 282 on the actuator 170 to hold the washer 278 in an axial position on the actuator. The spring 270 engages the washer 278 and urges the snap ring 280 into engagement with a shoulder 282 on the intermediate member 18. The spring 270 biases ithe actuator 170 in a distal direction. Accordingly, the spring 270 biases the handles 98 and 118 to pivot away from each other and the legs 222 and 226 to pivot toward each other.

Upon pivotal movement of the handles 98 and 118 toward each other, the actuator 170 moves in a proximal direction to compress the spring 270. The proximal movement of the actuator 170 causes the first ends 190 and 194 of the link members 186 and 188 to move in a proximal direction. Movement of the link members 186 and 188 in a proximal direction causes the legs 222 and 226 to pivot relative to the distal member 14 away from each other. When the handles 98 and 118 are released, the spring 270 moves the actuator 170 in a distal direction to pivot the legs 222 and 226 toward each other and the handles 98 and 118 away from each other.

The distance that the ends 234 and 252 of the legs 222 and 226 move away from each other is controlled by a stop member 272 (FIGS. 1-2) connected to the intermediate member 18 of the housing 12. The handles 98 and 118 engage the stop member 272 to limit the distance that the ends 234 and 252 move away from each other. The stop member 272 has a cylindrical main body portion 274 with an axially extending opening 276. The intermediate member 18 extends through the opening 276. The main body portion 274 may have any desired outer diameter.

A first projection 278 extends radially from the main body portion 274. A second projection 280 extends radially from an opposite side of the main body portion 274. The projections 278 and 280 may extend radially from the main body portion any desired distances. It is contemplated that the stop member 272 may have any number of radially extending projections.

A radially extending opening 282 (FIGS. 2 and 4) extends through the projection 280 and intersects the axially extending opening 276 in the main body portion 274. The radially extending opening 282 (FIG. 4) has a first radially outer portion 284 with a first diameter. The radially extending opening 282 has a second radially inner portion 286 with a second diameter smaller than the first diameter.

A guide pin member 288 extends through the radially extending opening 282. The pin member 288 is press fit into the second radially inner portion 286 of the opening 282. The pin member 288 has an end portion 290 that extends into a recess 292 in the intermediate member 18. The pin member 288 holds the stop member 272 in any one of a plurality of positions relative to the housing 12. The recess 292 (FIG. 2) in the intermediate member 18 has first and second axially extending portions 294 and 296. The first and second axially extending portions 294 and 296 are located 900 from each other. A circumferentially extending connecting portion 298 extends between proximal ends of the portions 294 and 296. The end portion 290 of the pin member 288 may be positioned in either one of the axially extending portions 294 and 296 of the recess 292. Although the recess 292 is shown as having two axially extending portions 294 and 296, it is contemplated that the recess may have any number of axially extending portions. Although the pin member 288 is shown extending from the stop member 272 into the recess 292 in the housing 12, it is contemplated that the guide member may extend from the housing into a recess in the stop member.

The handles 98 and 118 engage the stop member 272 to limit movement of the legs 222 and 226 away from each other. If the stop member 272 is in a first position on the intermediate member 18, as shown in FIG. 1, the handles 98 and 118 engage the main body portion 274 of the stop member to prevent further movement of the legs 222 and 226 away from each other. The legs 222 and 226 move away from each other a first predetermined distance. If the stop member 272 is in a second position on the intermediate member 18, the handles 98 and 118 engage the radially extending projections 278 and 280 of the stop member to prevent further movement of the legs 222 and 226 away from each other. The legs 222 and 226 move away from each other a second predetermined distance that is smaller than the first predetermined distance. When the stop member 272 is in the first position, the surgical tool 10 may be used to expand a tubular structure located adjacent a spine of a patient to perform a surgical procedure through the tubular structure to interconnect more than two vertebrae of the spine. When the stop member 272 is in the second position, the surgical tool 10 may be used to expand a tubular structure located adjacent a spine to perform a surgical procedure through the tubular structure to interconnect only two adjacent vertebrae.

When the pin member 288 extends into the axially extending portion 294, the stop member 272 is in the first position. When the pin member 288 extends into the axially extending portion 296, the stop member 272 is in the second position. The stop member 272 is moved between the first and second positions by moving the stop member axially in a proximal direction relative to the housing 12 until the pin member 288 extends into the connecting portion 298 of the recess 292. The stop member 272 is rotated 90° relative to the intermediate member 18 to align the pin member 288 with the desired axially extending portion 294 or 296. Once the pin member 288 is aligned with the desired axially extending portion 294 or 296, the stop member is moved axially in a distal direction relative to the housing 12. The pin member 288 holds the stop member 272 in the desired position relative to the housing 12.

Although the stop member 272 is described as having two positions relative to the intermediate member 18, it is contemplated that the stop member may have any desired number of positions relative to the intermediate member. The stop member 272 may have any desired number of projections extending radially from the main body portion 274. Each of the projections may extend a different distance from the main body portion 274 to limit the distance that the legs 222 and 226 move away from each other. Accordingly, the stop member 272 may define any number of predetermined distances that the legs 222 and 226 may move away from each other. It is also contemplated that the stop member 272 and/or the intermediate portion 18 may have markings to indicate the distance that the legs 222 and 226 may move away from each other.

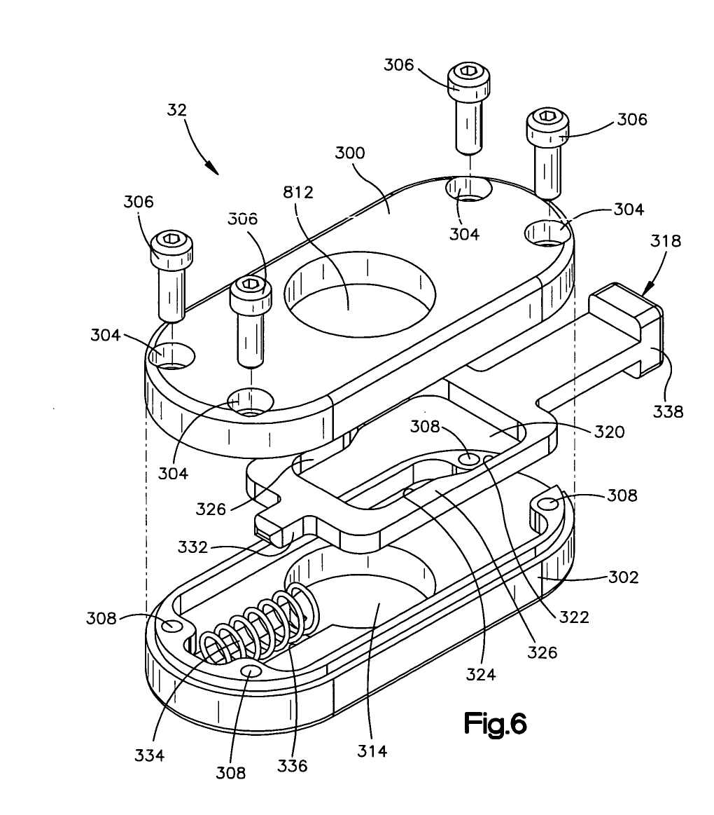

The depth limiter 32 (FIGS. 1-2 and 6) is positioned along the housing 12 to limit the depth that the surgical tool 10 may be inserted into the cannula or tubular structure. The depth limiter includes first and second housing members 300 and 302. The housing member 300 (FIG. 6) has four through-holes 304 through which screws 306 extend. The screws 306 threadably engage openings 308 in the second housing member 302 to connect the housing members 300 and 302 to each other.

The housing member 300 has a circular opening 312 through which the housing 12 extends. The housing member 302 has a circular opening 314 through which the housing 12 extends. The openings 312 and 314 have a diameter larger than the diameter of the housing 12.

A locking member 318 is slidably disposed within the housing members 300 and 302. The locking member 318 has an opening 320 which is aligned with the openings 312 and 314 in the housing members 300 and 302. The opening 320 has a first end 322 with a width greater than the diameter of the housing 12. Accordingly, when the end portion 322 of the opening 320 is aligned with the openings 312 and 314, the depth limiter 32 can move relative to the housing 12.

The opening 320 in the locking member 318 has a second end 324 with a width smaller than the diameter of the housing 12. The locking member 318 has side portions 326 that extend into the opening 320 to define the second end 324. The side portions 326 of the locking member 318 extend into the grooves 30 in the housing 12 to prevent movement of the depth limiter 32 relative to the housing 12.

The locking member 318 has a nose 332 that extends into a coil spring 334. The spring 334 biases the locking member 318 to align the second end 324 of the opening 320 with the openings 312 and 314 in the housing members 300 and 302. The spring 334 is located in recesses 336, one of which is shown in FIG. 6, in the housing members 300 and 302.

A button portion 338 of the locking member 318 extends out of the housing members 300 and 302. The button portion 338 may be depressed to move the locking member 318 so that the first end 322 of the opening 320 is aligned with the openings 312 and 314 to allow positioning of the depth limiter relative to the housing 12. Upon release of the button portion 338, the spring 334 moves the second end 324 of the opening 320 into alignment with the openings 312 and 314.

When the expansion tool 10 is to be inserted into the tubular structure or cannula, the depth limiter 32 is moved to a desired position along the housing 12 in accordance with the length of the cannula. The depth limiter 32 is positioned along the housing 12 so that the ends 234 and 252 of the legs 222 and 226 are located at a desired location in the tubular structure when the surgical tool 10 is inserted into the tubular structure. The stop member 272 is moved to a desired position in accordance with a desired amount of expansion of the tubular structure.

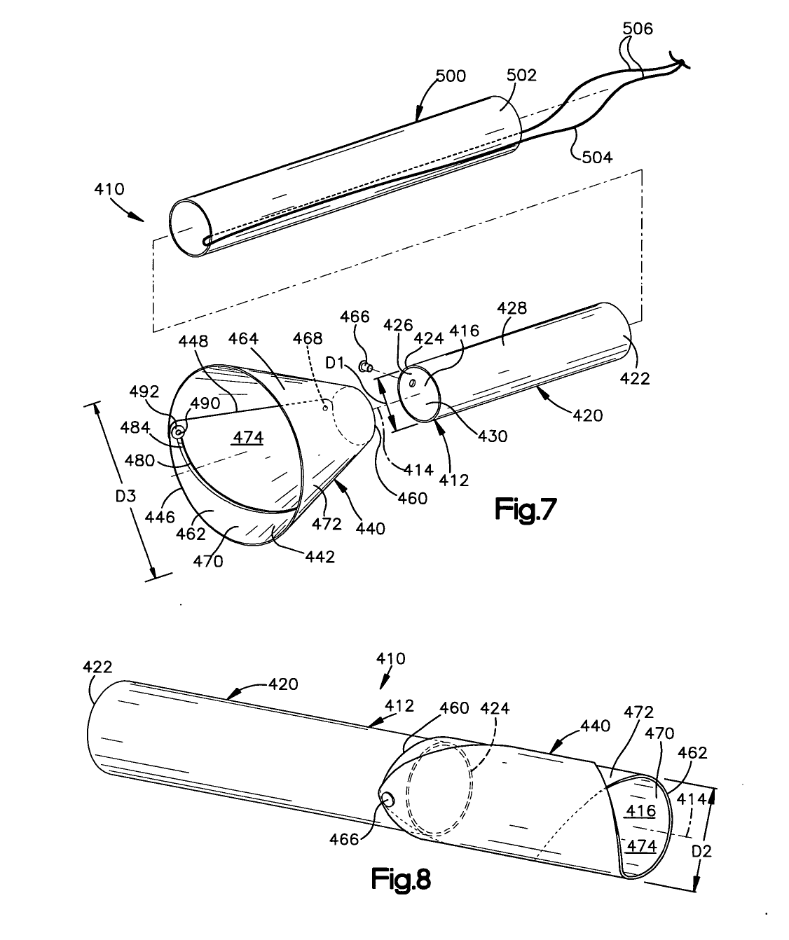

The surgical tool 10 may be used to expand any desired tubular structure, such as a tubular retractor or a cannula. One suitable tubular structure or cannula 410 is illustrated in FIGS. 7 and 8. The tubular structure 410 is suitable for use during a minimally invasive surgical procedure on a spine of a patent. The cannula 410 is a tubular structure 412 centered on an axis 414. The tubular structure 412 defines a passage 416 through the cannula 410. Surgical instruments are inserted into the body during a surgical procedure through the passage 416.

The tubular structure 412 comprises a first tubular portion 420 and a second tubular portion 440 attached to the first tubular portion. The first tubular portion 420 is may be made of any suitable material, such as a length of stainless steel tubing. The first tubular portion 420 has a proximal end 422 and a distal end 424. Parallel cylindrical inner and outer surfaces 426 and 428 (FIG. 7), respectively, extend between the ends 422, 424 of the first tubular portion 420. The inner surface 426 defines a first passage portion 430 of the passage 416 through the cannula 410. The first passage portion 430 has a diameter D1 which is preferably in the range from 10 mm to 30 mm or approximately 0.4 inches to approximately 1.2 inches.

The second tubular portion 440 of the tubular structure 412 is attached to the distal end 424 of the first tubular portion 420. The second tubular portion may be made of any suitable material, such as stainless steel. The second tubular portion 440 comprises an arcuate segment 442 of sheet stock. The arcuate segment 442 is rolled in an overlapping manner to form the tubular configuration of the second tubular portion 440. The second tubular portion 440 has first and second ends 460 and 462 connected by a central portion 464. The first end 460 of the second tubular portion 440 is attached to the distal end 424 of the first tubular portion 420 by a single suitable fastener 466, such as a rivet. The fastener 466 extends through two aligned apertures 468 at the first end 460 of the second tubular portion 440. The first end 460 of the second tubular portion 440 is pivotable about the fastener 466.

The second tubular portion 440 includes parallel inner and outer surfaces 470 and 472 (FIG. 7), respectively, extending between the first and second ends 460 and 462. The inner surface 470 defines a second passage portion 474 of the passage 416 through the cannula 410 which extends as a continuation of the first passage portion 430 in the first tubular portion 420.

An arcuate slot 480 is formed in the second tubular portion 440 and extends between the inner and outer surfaces 470 and 472 of the second tubular portion. The arcuate slot 480 extends along a curvilinear path in the central portion 464 of the second tubular portion 440 from the first end 462 toward the second end 460 of the second tubular portion. The arcuate slot 480 has a first terminal end (not shown) located in the central portion 464 of the second tubular portion 440. A second terminal end 484 of the arcuate slot 480 is located adjacent the intersection of an arcuate edge 446 and a first planar edge 448 of the arcuate segment 442.

A suitable guide member 490, such as guide pin, is attached to the inner surface 470 of the second tubular portion 440 adjacent the intersection of the arcuate edge 446 and a second planar edge (not shown) of the arcuate segment 442. In the tubular configuration of the second tubular portion 440, the guide member 490 is located in the arcuate slot 480 and is movable along the curvilinear path of the arcuate slot. A washer 492 is secured to an inner end of the guide member 490 to retain the guide member in the arcuate slot 480.

The second tubular portion 440 of the tubular structure 412 is expandable from a contracted condition shown in FIG. 8 to an expanded condition shown in FIG. 7. In the contracted condition, the guide member 490 is located in the first terminal end of the arcuate slot 480 in the second tubular portion 440 and the second passage portion 474 defined by the second tubular portion is cylindrical in shape. The second passage 474 has a generally constant diameter D2 (FIG. 8) which is approximately equal to the diameter D1 of the first tubular portion 420. Thus, the cross-sectional area of the second passage portion 474 at the second end 462 of the second tubular portion 440, which is a function of the diameter D2, is approximately the same as the cross-sectional area at the first end 460 of the second tubular portion and is approximately the same as the cross-sectional area of the first passage portion 430 in the first tubular portion 420.

In the expanded condition, shown in FIG. 7, the guide member 490 is located in the second terminal end 484 of the arcuate slot 480 in the second tubular portion 440 and the second tubular portion has a conical configuration. At the second end 462 of the second tubular portion 440, the second passage portion 474 has a diameter D3 (FIG. 7) which is larger than the diameter D2 of the second passage portion at the first end 460. Preferably, the diameter D3 of the second passage portion 474 at the second end 462 of the second tubular portion is 40% to 90% greater than the diameter D2 of the second passage portion at the first end 460. Thus, in the expanded condition, the cross-sectional area of the second passage portion 474 at the second end 462 of the second tubular portion 440, which is a function of the diameter D3, is greater than the cross-sectional area of the second passage portion at the first end 460 of the second tubular portion. Although the cross-sectional area at the second end 462 is shown as being circular in FIG. 7, it is contemplated that the cross-sectional area at the second end 462 could be any shape, such as oval shaped.

The cannula 410 includes an outer layer 500 (FIG. 7) for maintaining the second tubular portion 440 of the cannula in the contracted condition. It is contemplated that other suitable means for maintaining the second tubular portion 440 in the contracted condition could be employed. The outer layer 500 comprises a section of plastic tubing 502 which is heat shrunk over both the first and second tubular portions 420 and 440 to hold the second tubular portion in the contracted condition.

In addition, a loop of polyester string 504 for tearing the heat shrink tubing 502 is wrapped around the heat shrink tubing so that it extends both underneath and on top of the tubing. Outer ends 506 of the string 504 extend beyond the tubing 502.

During a minimally invasive surgical procedure, the cannula 410 is inserted through an incision into the body of a patient in the contracted condition. The cannula 410 is inserted through the incision using step dilation. The second tubular portion 440 is inserted inside the body. The first tubular portion 420 is inserted into the incision so that the first tubular portion extends from an exterior of the body to inside the body.

The outer ends 506 of the string 504 are then manually pulled on by the surgeon. Pulling on the string 504 tears the heat shrink tubing 502. With the heat shrink tubing 502 torn, the second tubular portion 440 of the cannula 410 is thereby released for expansion toward the expanded condition.

The expansion tool 10 is inserted into the passage 416 in the cannula 410 until the depth limiter 32 engages the tubular structure 412 and the ends 234 and 252 of the legs 222 and 226 are located at the second end 462 of the second tubular portion 440. The legs 222 and 226 of the tool 10 are separated by moving the handles 98 and 118 toward each other. As the handles 98 and 118 are moved toward each other, the ends 234 and 252 separate. The ends 234 and 252 move away from each other until the handles 98 and 118 engage the stop member 272. The maximum distance that the ends 234 and 252 move away from each other is determined by the position of the stop member 272.

As the ends 234 and 252 separate, a radially outwardly directed force is exerted on the inner surface 470 of the second tubular portion 440 by the ends 234 and 252, causing the second tubular portion to expand toward the expanded condition. Under the force of the expanding tool 10, the guide pin 490 slides from the first terminal end of the arcuate slot 480 toward the second terminal end 484 of the arcuate slot to permit the expansion of the second tubular portion 440. The tool 10 can be rotated about the axis 414 to ensure that the second tubular portion 440 of the cannula 410 is completely expanded to the desired expanded condition. The expansion tool 10 is then collapsed and removed so that one or more surgical instruments can be received through the cannula 10 and inserted into a patient's body.

The expandable second tubular portion 440 of the cannula 410 provides a significantly larger working area for the surgeon inside the body within the confines of the cannula. As a result, the simultaneous use of a number of surgical instruments, including but not limited to steerable instruments, shavers, dissectors, scissors, forceps, retractors, dilators, and endoscopes, is made possible by the expandable cannula 410.

It is contemplated that the surgical tool 10 could be used to expand any known tubular structure or cannula such as those described in U.S. Pat. Nos. 6,187,000 and 6,524,320 and U.S. Patent Application No. 10/361,887, filed Feb. 10, 2003, which are incorporated herein entirely by reference.

From the above description of the invention, those skilled in the art will perceive improvements, changes and modifications. Such improvements, changes and modifications within the skill of the art are intended to be covered by the appended claims.

1. A surgical tool for use in expanding a tubular structure, the tubular structure having an inner surface defining a passage through the tubular structure for receiving surgical instruments, said surgical tool comprising:
a housing;
a first leg movable relative to said housing having a first end engageable with the inner surface of the tubular structure;
a second leg movable relative to said housing having a second end engageable with the inner surface of the tubular structure;
a handle movable relative to said housing to move said first and second ends away from each other to apply a radially outwardly directed force to the inner surface of the tubular structure and cause expansion of the tubular structure to increase a cross-sectional area of the passage along a portion of the passage; and
a stop member connected with said housing, said handle being engageable with said stop member to limit movement of said first and second ends away from each other.

2. A surgical tool as set forth in claim 1 wherein said stop member is positionable relative to said housing, said stop member having a first position relative to said housing to limit movement of said first and second ends away from each other a first predetermined distance, said stop member having a second position relative to said housing to limit movement of said first and second ends away from each other a second predetermined distance smaller than the first distance.

3. A surgical tool as set forth in claim 2 wherein said stop member includes first and second portions, said handle being engageable with said first portion when said stop member is in said first position, said handle being engageable with said second portion of said stop member when said stop member is in said second position.

4. A surgical tool as set forth in claim 2 wherein said stop member includes a main body portion and a projection extending radially from said main body portion, said handle being engageable with said main body portion when said stop member is in said first position, said handle being engageable with said projection when said stop member is in said second position.

5. A surgical tool as set forth in claim 2 wherein one of said stop member and said housing includes a guide member extending into a recess in the other one of said stop member and said housing.

6. A surgical tool as set forth in claim 5 wherein said recess includes a plurality of axially extending portions and a connecting portion extending between said axially extending portions.

7. A surgical tool as set forth in claim 6 wherein said stop member is rotatable and axially movable relative to said housing.

8. A surgical tool as set forth in claim 1 including first and second handles movable relative to said housing to move said first and second ends away from each other, said first and second handles being engageable with said stop member to limit movement of said first and second ends away from each other.

9. A surgical tool as set forth in claim 8 wherein said first and second handles are movable toward each other to move said first and second ends away from each other.

10. A surgical tool as set forth in claim 1 further including an axially movable actuator, said handle being movable relative to said housing to axially move said actuator relative to said housing and move said first and second ends away from each other.

11. A surgical tool as set forth in claim 1 further including a depth limiter for limiting the depth that said surgical tool extends into the passage in the tubular structure.

12. A surgical tool as set forth in claim 1 wherein said first and second legs are pivotable relative to each other.

13. A surgical tool for use in expanding a tubular structure, the tubular structure having an inner surface defining a passage through the tubular structure for receiving surgical instruments, said surgical tool comprising:
a housing;
a first leg movable relative to said housing having a first end engageable with the inner surface of the tubular structure;
a second leg having a second end engageable with the inner surface of the tubular structure, said first leg being movable relative to said second leg;
a handle movable relative to said housing to move said first end away from said second end to apply a radially outwardly directed force to the inner surface of the tubular structure and cause expansion of the tubular structure to increase a cross-sectional area of the passage along a portion of the passage; and
a stop member connected with said housing, said handle being engageable with said stop member to limit movement of said first end away from said second end, said stop member being positionable relative to said housing, said stop member having a first position relative to said housing to limit movement of the first end away from the second end a first predetermined distance, said stop member having a second position relative to said housing to limit movement of the first end away from the second end a second predetermined distance smaller than the first predetermined distance.

14. A surgical tool as set forth in claim 13 wherein said stop member includes first and second portions, said handle being engageable with said first portion when said stop member is in said first position, said handle being engageable with said second portion of said stop member when said stop member is in said second position.

15. A surgical tool as set forth in claim 13 wherein said stop member includes a main body portion and a projection extending radially from said main body portion, said handle being engageable with said main body portion when said stop member is in said first position, said handle being engageable with said projection when said stop member is in said second position.

16. A surgical tool as set forth in claim 13 wherein one of said stop member and said housing includes a guide member extending into a recess in the other one of said stop member and said housing.

17. A surgical tool as set forth in claim 16 wherein said recess includes a plurality of axially extending portions and a connecting portion extending between said axially extending portions.

18. A surgical tool as set forth in claim 17 wherein said stop member is rotatable and axially movable relative to said housing.

19. A surgical tool as set forth in claim 13 including first and second handles movable relative to said housing to move said first and second ends away from each other, said first and second handles being engageable with said stop member to limit movement of said first end away from said second end.

20. A surgical tool as set forth in claim 19 wherein said first and second handles are movable toward each other to move said first and second ends away from each other.

21. A surgical tool as set forth in claim 13 further including an axially movable actuator, said handle being movable relative to said housing to axially move said actuator relative to said housing and move said first and second ends away from each other.

22. A surgical tool as set forth in claim 13 further including a depth limiter for limiting the depth that said surgical tool extends into the passage in the tubular structure.

23. A surgical tool as set forth in claim 13 wherein said first and second legs are pivotable relative to each other.
